(19) United States
(12) Patent Application Publication
(10) Pub. No.: US 2022/0395171 A1
(43) Pub. Date: Dec. 15, 2022

(54) SURGICAL CAMERA SYSTEM

(71) Applicant: ARTHREX, INC., Naples, FL (US)

(72) Inventors: Shane Noble, Naples, FL (US); Josh Karnes, Estero, FL (US)

(21) Appl. No.: 17/348,638

(22) Filed: Jun. 15, 2021

(51) Int. Cl. A61B 1/313; A61B 1/00; A61B 1/05; A61B 1/07

(52) U.S. Cl. CPC A61B 1/313; A61B 1/00066; A61B 1/05; A61B 1/00177; A61B 1/07; A61B 1/0008; A61B 1/3135

(57) ABSTRACT

A camera device having a handpiece; a shaft having a proximal end coupled to the handpiece; and a distal end; and a probe coupled to the distal end of the shaft, the probe having a proximal portion extending from the distal end of the shaft along a longitudinal axis, and a distal portion extending at an angle of between about 70 degrees and about 110 degrees relative to the longitudinal axis, the distal portion having an image sensor assembly.

### BACKGROUND

The present disclosure relates to devices used in surgery and, more particularly, to a camera and probe.

Typically, during spinal surgery, in order to view a lateral recess, neural foramen and spinal canal to assess proper decompression due to a sequestered disc fragment or spinal stenosis, the boney structure covering the anatomy is removed. This process is referred to as “unroofing”. The removal of the boney structure may lead to destabilization of the spine. Additionally, if too much bone is removed during the “unroofing” process, the spine may become so unstable that the patient requires the spine to be artificially stabilized, such as through a spinal fusion procedure, to maintain stability of the spine.

Therefore, there exists a need for a system and method of providing direct visualization during spinal surgery that remedies the shortcomings of the prior art.

### SUMMARY

The present invention is directed to a camera device that provides for direct visualization during spinal surgery that allows a user to better assess a patient's anatomy without unroofing. In an implementation, a camera device has a handpiece; a shaft further comprising: a proximal end coupled to the handpiece; and a distal end. The camera device also has a probe coupled to the distal end of the shaft, the probe having: a proximal portion extending from the distal end of the shaft along a longitudinal axis; and a distal portion extending at an angle of between about 70 degrees and about 110 degrees relative to the longitudinal axis, the distal portion having an image sensor assembly.

The distal portion of the probe may extend at an angle of between about 85 degrees and about 95 degrees relative to the longitudinal axis. Optionally, the shaft has a diameter of less than about 2 mm. Optionally, the shaft has a length of less than about 150 mm. The probe may have a bend between the proximal portion and the distal portion; and the bend may have a radius of between about 1.9 mm and about 7.6 mm.

The probe may further have a printed wiring assembly extending from the image sensor assembly to the handpiece. Additionally, the handpiece may have at least one light source; and a plurality of optical fibers that extend from the at least one light source to the distal portion of the probe.

In an implementation, the proximal portion of the probe has a diameter of between about 3 mm and about 6 mm and a length of between about 4 mm and about 15 mm. Optionally, the distal portion of the probe has a diameter of between about 5 mm and about 10 mm and a length of between about 3.8 mm and about 15 mm. In an implementation, the distal portion of the probe has a length of between about 5 mm and about 6.5 mm and a diameter of between about 6.6 mm and about 7.1 mm. In an implementation, the distal portion of the probe further comprises a wall thickness greater than about 1.7 mm.

The distal portion of the probe may have a bumper. The bumper may have a thickness of between about 2 mm and about 10 mm. The distal portion of the probe may have a ball. The ball may have a diameter of between about 3 mm and about 10 mm. In an implementation, the probe is formed of two halves that are coupled together around at least a portion of the shaft. The probe may be made of plastic. The shaft may have at least one bend.

The present invention is also directed to a method of using a camera device, wherein the camera device has a handpiece; a shaft further comprising: a proximal end coupled to the handpiece, a distal end, and a longitudinal axis from the proximal end to the distal end; and a probe coupled to the distal end of the shaft, the probe further comprising: a proximal portion extending along the longitudinal axis, and a distal portion extending at an angle of between about 70 degrees and about 110 degrees relative to the longitudinal axis. The method has the steps of: inserting the camera device into a patient to a desired surgical site; using the probe to move tissue out of a field of view; imaging the surgical site; and withdrawing the camera device from the patient.

In an implementation, a camera device has a handpiece with a light source and a shaft with: a proximal end coupled to the handpiece; and a distal end. A probe is coupled to the distal end of the shaft, the probe having: a proximal portion extending from the distal end of the shaft along a longitudinal axis, the proximal portion having a diameter of between about 3 mm and about 6 mm and a length of between about 4 mm and about 15 mm. The probe also has a distal portion extending at an angle of between about 70 degrees and about 110 degrees relative to the longitudinal axis. The distal portion having a diameter of between about 6 mm and about 15 mm; a length of between about 3.8 mm and about 15 mm; and an image sensor assembly. A printed wiring assembly extends from the image sensor assembly to the handpiece and a plurality of optical fibers extend from the light source to the distal portion of the probe.

These and other features are described below.

### BRIEF DESCRIPTION OF THE DRAWINGS

The features, aspects and advantages of the present invention will become better understood with regard to the following description, appended claims and accompanying figures wherein:

FIG. 1 is a schematic diagram of a camera system according to an implementation of the present invention;

FIG. 2 is a side elevation view of a camera device according to an implementation;

FIG. 3 is a cross sectional side elevation view of the device of FIG. 2;

FIG. 4 is a side perspective elevation view of a probe according to an implementation;

FIG. 5 is a side perspective elevation view of a probe according to an implementation;

FIG. 6 is a cross sectional perspective elevation view of a first portion of the probe of FIG. 4 without a shaft and internal components;

FIG. 7 is a cross sectional perspective elevation view of a second portion of the probe of FIG. 4 without a shaft and internal components;

FIG. 8 is a side perspective elevation view of a camera device with a shaft having a bend according to an implementation; and

FIG. 9 is a side perspective elevation view of a camera device with a shaft having two bends according to an implementation.

### DETAILED DESCRIPTION

In the following description of the preferred implementations, reference is made to the accompanying drawings which show by way of illustration specific implementations in which the invention may be practiced. Wherever possible, the same reference numbers will be used throughout the drawings to refer to the same or like parts. It is to be understood that other implementations may be utilized and structural and functional changes may be made without departing from the scope of this disclosure.

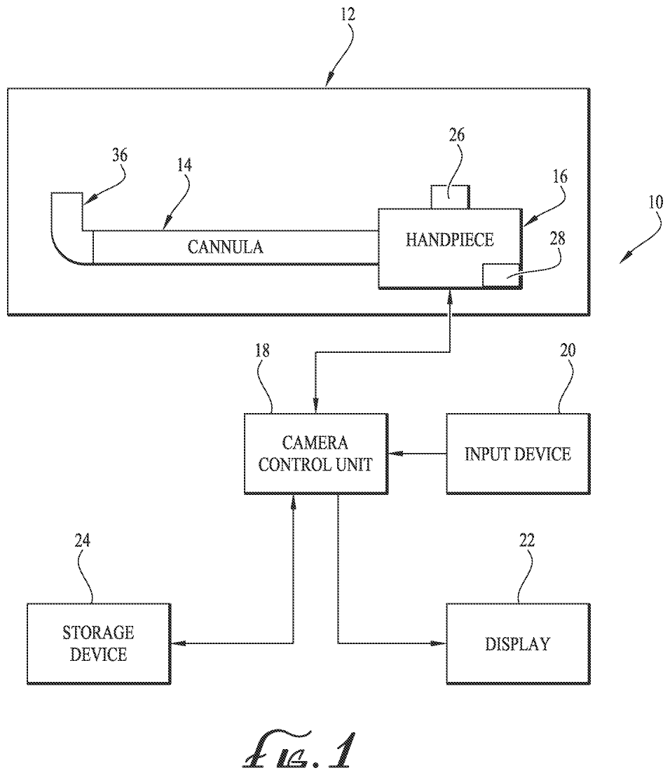

With reference to FIG. 1, a camera system 10 according to an implementation has a surgical camera probe 12 with at least one cannula 14 coupled to a handpiece 16. The handpiece 16 is connectable to a camera control unit 18 (“CCU”). The handpiece 16 and camera control unit 18 may be connected via wire or wirelessly. The camera control unit 18 is also connectable to at least one input device 20 such as a mouse, keyboard, touchpad, or touchscreen monitor. Additionally, the camera control unit is connectable to a display 22 and a storage device 24, such as an external hard drive or server. In implementations, the surgical camera probe 12 may be used in endoscopic surgery.

The handpiece 16 may have memory for storing camera data, camera control unit processing data or other information. The handpiece 16 may also have a user input means 26 such as buttons to control aspects of image capture. The handpiece 16 may also have a light source 28, such as a light emitting diode.

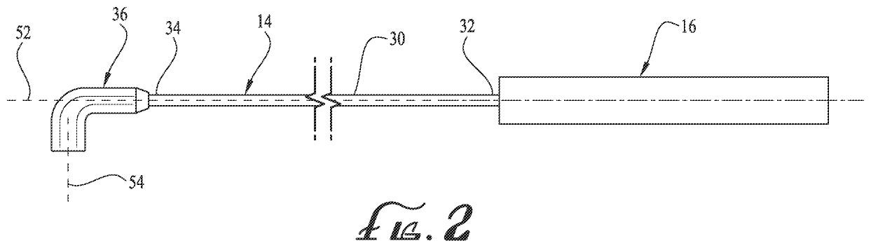

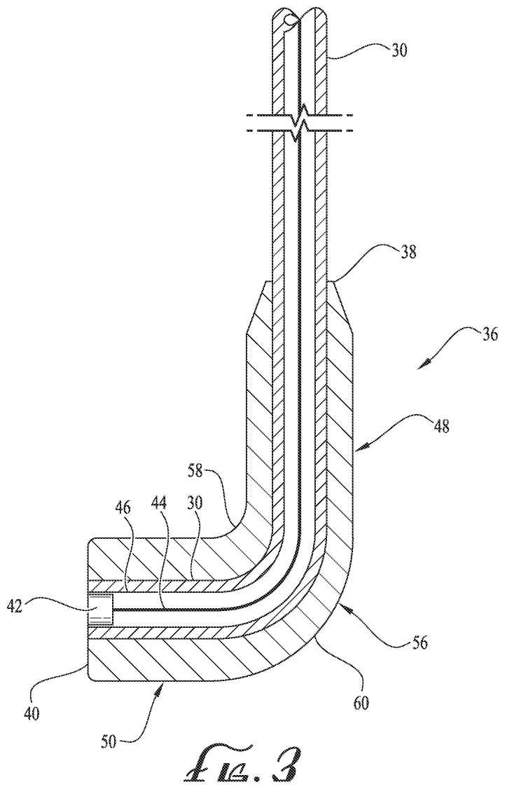

With reference to FIGS. 2 and 3, the surgical camera probe 12, according to an implementation, will now be described in further detail. The cannula 14 has a shaft 30 with a diameter of less than about 20 mm, preferably less than about 2.0 mm and more preferably less than about 1.9 mm. The shaft 30 is less than about 450 mm long, and more preferably less than about 150 mm long. A proximal end 32 of the shaft 30 is coupled to the handpiece 16. A distal end 34 of the shaft 30 is coupled to a probe 36. In an implementation, a portion of the shaft 30 extends into the probe 36.

With reference to FIG. 3, the probe 36 has a proximal end 38 coupled to the shaft 30 and a distal end 40. In an implementation, an image sensor assembly 42 is positioned proximal to the distal end 40 of the probe 36. As used herein, the term image sensor assembly includes an image sensor, such as, for example, a complementary metal-oxide-semiconductor (CMOS) sensor or charge-coupled device (CCD) sensor. The image sensor assembly 38 may contain a plurality of image sensors. The image sensor assembly 42 may contain sensors capable of sensing visible light and may contain sensors capable of sensing non-visible light, such as near infra-red (NIR) light. Additionally, the image sensor assembly 42 may include associated optics, such as, for example, lenses, filters and prisms; and may also include operational circuitry. The image sensor assembly 42 is configured for front viewing out of the distal end 36 of the probe 32.

The image sensor assembly 42 is electrically coupled to the handpiece. In an implementation, a printed wiring assembly (PWA) 44 extends from the image sensor assembly 42, through the shaft 30 to a printed circuit board in the handpiece 16. Alternatively, the image sensor assembly 42 may be coupled to a wireless data transmitter that communicates image data to an image data receiver in the handpiece 16 or camera control unit 18.

In an implementation, a bundle of optical fibers 46 extends from the light source 28 in the handpiece 16 along the shaft 30, through the probe 36, and terminates proximal to the distal end 40 of the probe. The optical fibers 46 transmit light from the light source 28 out of the distal end 40 of the probe 36 to illuminate a surgical scene. In alternative implementations, a light source or plurality of light sources may be positioned proximal to the distal end 40 of the probe 36. Power may be provided to the light source or plurality of light sources using a wire extending from the handpiece 16 along the shaft 30. The light source 28 may emit white light and may emit light of other wavelengths, such as, for example, ultraviolet and infra-red.

With reference to FIG. 3, the probe 36 according to an implementation has a proximal portion 48, and a distal portion 50 positioned at an angle relative to the proximal portion. In an implementation, the angle between the proximal portion 48 and the distal portion 50 is between about 70 degrees and about 110 degrees, and more preferably between about 80 degrees and about 100 degrees, and more preferably between about 85 degrees and about 95 degrees. In an implementation, the angle between the proximal portion 48 and the distal portion 50 is about 90 degrees.

The proximal portion 48 of the probe 36 extends from the distal end 34 of the shaft 30 along a longitudinal axis 52. The distal portion 52 extends along a view axis 54. The angle between the longitudinal axis 52 and the view axis 54 is between about 70 degrees and about 110 degrees, and more preferably between about 80 degrees and about 100 degrees, and more preferably between about 85 degrees and about 95 degrees. In an implementation, the angle between the longitudinal axis 52 and the view axis 54 is about 90 degrees.

In an implementation, the proximal portion 48 and the distal portion 50 are separated by a bend 56 having a radius of between about 1.2 mm and about 10 mm, and more preferably between about 1.9 mm and about 7.6 mm. In an implementation, the bend 56 has an inner radius 58 on an inside of the bend and an outer radius 60 on an outside of the bend. In an implementation, the inner radius 58 is between about 2.4 mm and about 2.5 mm and the outer radius 60 is between about 6.9 mm and about 7.1 mm.

The proximal portion 48 has a diameter of between about 3 mm and about 6 mm. In an implementation, the proximal portion 48 tapers from about 3.1 mm at the proximal end 38 to a diameter of about 5 mm. The proximal end 38 of the probe 36 has an opening about 2.8 mm and is configured to fit around the shaft 30. The proximal portion 48 has a length of between about 4 mm and about 15 mm, and more preferably between about 5 mm and about 10 mm.

The distal portion 50 has a length of between about 3.8 mm and about 15 mm, and more preferably between about 5 mm and about 6.5 mm. The distal portion has an outer diameter of between about 5 mm and about 10 mm, and more preferably between about 6.3 mm and about 7.6 mm, and more preferably between about 6.6 mm to about 7.1 mm.

The diameter of the distal portion 50 allows for a wall thickness of greater than about 1.7 mm around the shaft 30 which creates a rigid structure that can be used to move tissue out of the field of view. For example, the camera may be used to move tissue surrounding the spine out of the field of view and allow for imaging of the lateral recess, neural foramen and spinal canal of a human. Preferably, the shaft 30 and probe 36 are configured to reach a lumbar region of the spine from a posterior approach.

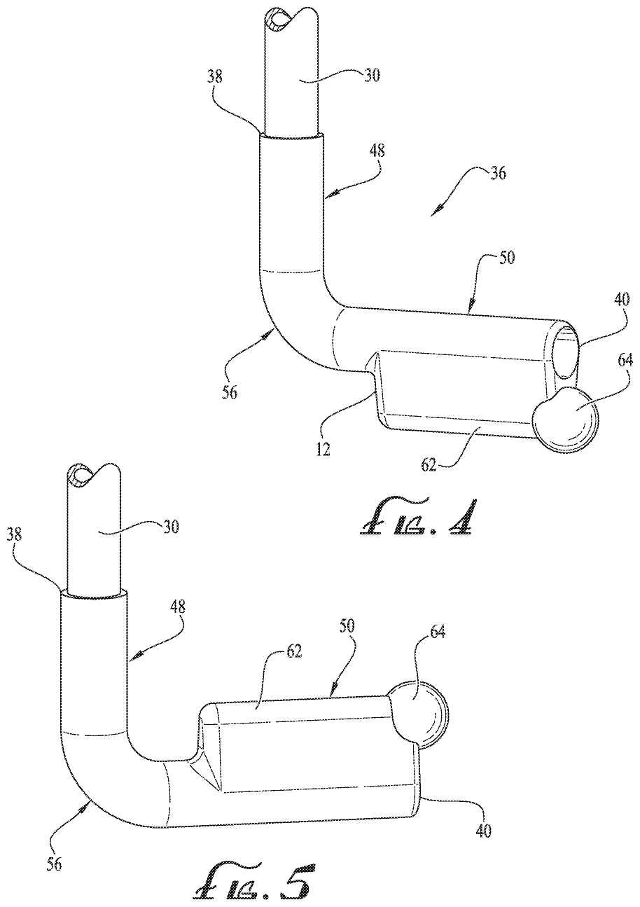

In additional implementations of the invention as shown in FIGS. 4 and 5, the probe 36 may further comprise a bumper 62 and a ball 64 on the distal portion 50. The bumper 62 may have a thickness of between about 2 mm and about 10 mm, and more preferably between about 3 mm and about 6 mm. The ball 64 may have a diameter of between about 3 mm and about 10 mm, and more preferably between about 4 mm and about 6 mm. The ball 64 is positioned proximal to the distal tip 40. As shown in FIG. 4, the bumper 62 and the ball 64 may be positioned outside of the image sensor assembly 42 to assist in moving tissue away from the field of view. As used herein, the phrase “outside of the image sensor assembly” refers to a position that is outside of the bend 56 and farther from the handpiece 16 than the image sensor assembly 42.

As shown in FIG. 5, the bumper 62 and the ball 64 may be positioned inside of the image sensor assembly 42 to assist in moving tissue away from the field of view. As used herein, the phrase “inside of the image sensor assembly” refers to a position that is on inside of the bend 56 and closer to the handpiece 16 than the image sensor assembly 42.

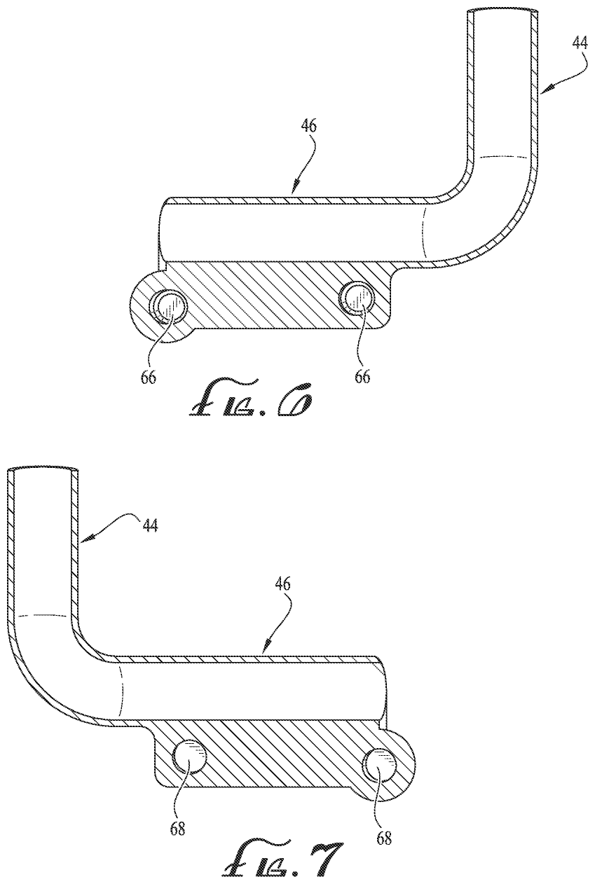

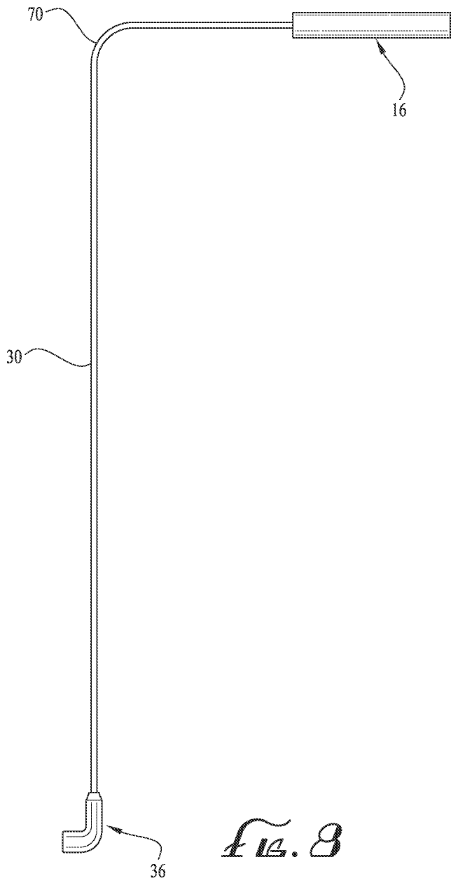

The probe 36 may be made from plastic, such as, for example, acrylonitrile butadiene styrene (ABS) plastic. The probe 36 may be made from other rigid materials, such as and without limitation: polyether ether ketone (PEEK), polycarbonate, high density polyethylene (HDPE), and metals such as stainless steel, aluminum, titanium, and brass. With reference to FIGS. 6 and 7, the probe 36 may be made using two mirrored halves that are assembled around portions of the shaft 30. The probe halves may be attached to each other using, for example and without limitation, adhesive, ultrasonic welding or a press fit. As shown in FIGS. 6 and 7, a first half of the probe 36 may have a plurality of pins 66 that press fit into a plurality of slots 68 to hold the probe together. Additionally, an adhesive may be placed inside of the probe 32 to hold portions of the shaft 30 in place inside the probe 32. For example, and without limitation, an adhesive that may be used is Loctite #4041.

The probe 36 allows a user (such as a surgeon) to move tissue surrounding the spine out of the field of view of the image sensor assembly 42. This allows a user to better assess the anatomy before determining the need to destabilize the spine by unroofing the affected anatomy. This can lead to better surgical outcomes by minimizing the need for unneeded fusion procedures.

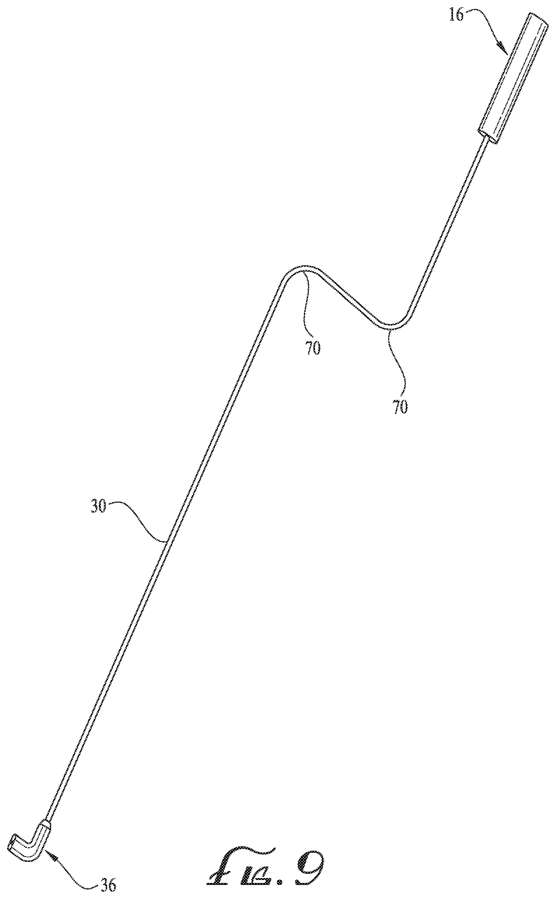

In additional implementations, as shown in FIGS. 8 and 9 the shaft 30 has one or more bends 70 to allow a user to more easily access different parts of a patient's anatomy. As shown in FIG. 8, the shaft 30 may have a single bend 70, As shown in FIG. 9, the shaft 30 may have two different bends 70. Each bend may be about 90 degrees. Additionally, each bend may be between about 30 degrees and about 150 degrees, and more preferably between about 45 degrees and about 135 degrees, and more preferably between about 70 degrees and about 110 degrees. The shaft 30 may be pre-bent with different configurations. Additionally, a user may manually bend the shaft at the time of use depending on, for example and without limitation, the anatomy of the patient and the procedures to be performed.

An exemplary use of the surgical camera system will now be described. Initially, an incision is made in a back of a patient proximal to the spine and a dilator is introduced, A portal is then inserted through the dilator and into a spinal column of the patient. The portal may have a diameter of between about 14 mm and about 22 mm. The portal may have a length of between about 40 mm to about 120 mm depending on the anatomy of the patient. The camera 12 is then inserted into the portal and the probe 36 used to manipulate tissue in the spinal column to allow for imaging of the spinal column area. The camera may be used to image the lateral recess, neural foramen and spinal canal.

There is disclosed in the above description and the drawings, a surgical camera system and method that fully and effectively overcomes the disadvantages associated with the prior art. However, it will be apparent that variations and modifications of the disclosed implementations may be made without departing from the principles of the invention. The presentation of the implementations herein is offered by way of example only and not limitation, with a true scope and spirit of the invention being indicated by the following claims.

Any element in a claim that does not explicitly state “means” for performing a specified function or “step” for performing a specified function, should not be interpreted as a “means” or “step” clause as specified in 35 U.S.C. § 112.

What is claimed is:

1. A camera device comprising:
a handpiece;
a shaft further comprising:
a proximal end coupled to the handpiece; and
a distal end;
a probe coupled to the distal end of the shaft, the probe further comprising:
a proximal portion extending from the distal end of the shaft along a longitudinal axis; and
a distal portion extending at an angle of between about 70 degrees and about 110 degrees relative to the longitudinal axis, the distal portion further comprising an image sensor assembly.

2. The camera device of claim 1 wherein the distal portion of the probe extends at an angle of between about 85 degrees and about 95 degrees relative to the longitudinal axis.

3. The camera device of claim 1 wherein the shaft further comprises a diameter of less than about 2 mm.

4. The camera device of claim 3 wherein the shaft further comprises a length of less than about 150 mm.

5. The camera device of claim 1 wherein the probe further comprises a bend between the proximal portion and the distal portion; and wherein the bend further comprises a radius of between about 1.9 mm and about 7.6 mm.

6. The camera device of claim 1 wherein a printed wiring assembly extends from the image sensor assembly to the handpiece.

7. The camera device of claim 1 wherein the handpiece further comprises at least one light source; and wherein a plurality of optical fibers extend from the at least one light source to the distal portion of the probe.

8. The camera device of claim 1 wherein the proximal portion of the probe further comprises a diameter of between about 3 mm and about 6 mm and a length of between about 4 mm to about 15 mm.

9. The camera device of claim 1 wherein the distal portion of the probe further comprises a diameter of between about 5 mm and about 10 mm and a length of between about 3.8 mm and about 15 mm.

10. The camera device of claim 9 wherein the distal portion of the probe further comprises a length of between about 5 mm and about 6.5 mm and a diameter of between about 6.6 mm and about 7.1 mm.

11. The camera device of claim 1 wherein the distal portion of the probe further comprises a wall thickness greater than about 1.7 mm.

12. The camera device of claim 1 wherein the distal portion of the probe further comprises a bumper.

13. The camera device of claim 12 wherein the bumper further comprises a thickness of between about 2 mm and about 10 mm.

14. The camera device of claim 1 wherein the distal portion of the probe further comprises a ball.

15. The camera device of claim 14 wherein the ball further comprises a diameter of between about 3 mm and about 10 mm.

16. The camera device of claim 1 wherein the probe further comprises two halves that couple together around at least a portion of the shaft.

17. The camera device of claim 1 wherein the probe further comprises plastic.

18. The camera device of claim 1 wherein the shaft further comprises at least one bend.

19. A method of using a camera device, wherein the camera device comprises a handpiece; a shaft further comprising: a proximal end coupled to the handpiece; a distal end; and a longitudinal axis from the proximal end to the distal end; and a probe coupled to the distal end of the shaft, the probe further comprising: a proximal portion extending along the longitudinal axis; and a distal portion extending at an angle of between about 70 degrees and about 110 degrees relative to the longitudinal axis, the method comprising:
inserting the camera device into a patient to a desired surgical site;
using the probe to move tissue out of a field of view;
imaging the surgical site; and
withdrawing the camera device from the patient.

20. A camera device comprising:
a handpiece further comprises a light source;
a shaft further comprising:
a proximal end coupled to the handpiece;
a distal end; and
a probe coupled to the distal end of the shaft, the probe further comprising:
a proximal portion extending from the distal end of the shaft along a longitudinal axis, the proximal portion further comprising a diameter of between about 3 mm and about 6 mm and a length of between about 4 mm and about 15 mm; and
a distal portion extending at an angle of between about 70 degrees and about 110 degrees relative to the longitudinal axis, the distal portion further comprising:
a diameter of between about 6 mm and about 15 mm;
a length of between about 3.8 mm and about 15 mm; and
an image sensor assembly;
wherein a printed wiring assembly extends from the image sensor assembly to the handpiece and a plurality of optical fibers extend from the light source to the distal portion of the probe.
